(19) United States
(12) Patent Application Publication
(10) Pub. No.: US 2023/0181304 A1
(43) Pub. Date: Jun. 15, 2023

(54) IMPLANT

(71) Applicant: OLYMPUS WINTER & IBE GMBH, Hamburg (DE)

(72) Inventors: Thorsten JÜRGENS, Hamburg (DE); Thomas WOSNITZA, Luneburg (DE); Henning SCHWEINBERGER, Basthorst (DE)

(73) Assignee: OLYMPUS WINTER & IBE GMBH, Hamburg (DE)

(21) Appl. No.: 18/078,607

(22) Filed: Dec. 9, 2022

Related U.S. Application Data
(60) Provisional application No. 63/289,829, filed on Dec. 15, 2021.

(51) Int. Cl. A61F 2/04

(52) U.S. Cl. CPC A61F 2/04; A61F 2002/047; A61F 2210/0014

(57) ABSTRACT

An implant with which a urinary tract can be treated in a simple and reliable manner is achieved in that a wire structure of an implant can be introduced into the urethra in a folded state with a distal end in the lead. The wire structure is unfolded inside the urethra for the treatment of the tissue. The ischemic pressure of the wires on the surrounding tissue results in denaturation. At least one further device for constricting or cutting into tissue is positioned on the wire structure. This at least one further device is designed in such a way that it exerts a local ischemic pressure on the median lobe in the unfolded state of the implant.

The invention relates to an implant in accordance with the preamble of claim 1.

Various methods and techniques are known for the treatment of the urinary tract, in particular a benign prostatic enlargement (benign prostatic hyperplasia, BPH). In a minimally invasive, particularly gentle treatment of BPH symptoms, a removable implant is temporarily placed in the urethra or in the prostatic portion of the urethra of the patient. Such an implant is a wire structure made of a shape memory alloy, such as, for example, Nitinol. In a folded state, the wire structure is pushed into the correct position by a catheter in order to unfold there into its predetermined basic structure. This structure, which can be formed from three or four wires, is a basket structure. This basket structure widens the urethra. Owing to the expansion of the wire structure against the tissue of the urethra, the tissue of the urethra is denatured over the course of a few days. This denaturation of the tissue takes place on account of the ischemic pressure of the individual wires on the cells of the tissue, leading to reduced or completely absent blood flow. As a result, the lack of blood supply leads to a lack of oxygen in the cells and ultimately to death of the cells. Within a few days, the tissue can be reduced to such an extent that the urinary flow almost normalizes. After completion of this treatment, the implant can be recovered from the urethra by means of a catheter.

By means of the technique described above, the tissue in the urethra can be treated in a very efficient manner. Owing to the enlargement of the prostate, the Lobus medius (median lobe) may also protrude a long way into the interior of the bladder. It is conceivable that the bladder outlet will be displaced as a result of this protrusion of the median lobe, but also as a result of a possible enlargement of the median lobe. However, this displacement of the bladder outlet by protrusion and/or by enlargement of the median lobe can also occur independently of an enlargement of the prostate. For medical reasons, it is important to avoid displacement of the bladder outlet. However, known methods and techniques for this are rather complex and traumatizing for the patient. The technique described above is also not suitable for treating the urinary tract in an appropriate way.

Proceeding therefrom, the object of the invention is that of providing an implant with which the urinary tract can be treated in a simple and reliable manner.

One way of achieving this object is described by the features of claim 1. Accordingly, it is envisaged that an implant, in particular a removable implant, which consists of a wire structure with at least two wires, can be introduced into the urethra of a patient in a folded state with a distal end in the lead. Within the urethra, the wire structure is unfolded for the treatment of the tissue. The ischemic pressure of the wires on the surrounding tissue results in the denaturation described above. The invention envisages that at least one further means for constricting or cutting into tissue is positioned on the wire structure. This at least one further means is designed in such a way that it exerts a local ischemic pressure on the median lobe in the unfolded state of the implant. Thus, in addition to the treatment of the urethra tissue by the further means, the median lobe is also treated. After completion of the treatment, the tissue of the median lobe is denatured owing to the continuous pressure. The denaturation of the tissue has the effect that space is created for the continued flow of urine. Owing to the fact that this further means is arranged on a wire structure for the treatment of BPH symptoms, the urinary tract can be treated in a particularly efficient, simple and gentle manner.

The invention preferably provides for the means for constricting or cutting to be a wire made from a shape memory material or to be a thread. The use of a shape memory material, such as, for example, Nitinol, proves to be particularly advantageous since the shape of the means or the relative alignment of the means with respect to the wire structure can be predetermined. Thus, after insertion into the body through a catheter, the implant unfolds back into the predetermined initial structure. This initial structure is optimally adapted to the given anatomical conditions, thereby enabling particularly efficient treatment of the tissue to be achieved.

The use of a thread proves to be particularly simple and advantageous technically. Thus, for example, a thread can be tied in any desired shape around the tissue and removed again after completion of the treatment. Moreover, a simple thread is particularly space-saving, this being extremely relevant particularly for minimally invasive treatments.

In particular, the present invention furthermore envisages that the wire can be introduced into the urethra in a folded state relative to the implant and unfolds in the urethra so as to form a predetermined angle with the wire structure. This angle is such that the implant reaches the urethra just as much as the median lobe. Moreover, the angle is such that the wire presses against the tissue of the median lobe with a mechanical prestress. The lateral alignment of the wire relative to the wire structure can also be defined in this way. It has proven to be particularly advantageous if the additional wire lies on the same longitudinal axis as the wire structure. By means of this relative orientation, the implant can be positioned in a very precise manner and can be removed or pulled into the catheter after treatment.

As a preferred option, it is furthermore conceivable that the means for constricting or cutting, in particular the wire or the thread, is intertwined with a wire of the wire structure. A sufficiently firm connection can be produced by means of this intertwining of the wire or the thread with the wire structure. In addition, this means that no further fastening means are necessary in order to connect the further means to the wire structure. As a result of this intertwining, the further means is coupled to the wire structure in terms of movement, and it is thereby possible to achieve the specified combined treatment of the urethra and the median lobe.

It is furthermore conceivable, according to the invention, that the wire is bent at least in some section or sections, wherein the wire is bent convexly relative to a longitudinal axis of the implant, thus enabling the bent wire to be laid around the median lobe. As a result of the described shape of the wire, the wire can exert pressure on the median lobe over its entire length. As a result, the treatment or denaturing of the tissue can be accomplished in a very efficient manner. By virtue of this shape, it is also possible to cut the tissue in a particularly efficient way since a preload is also exerted on the tissue on account of the convex shape.

A further advantageous exemplary embodiment of the invention can make provision for the thread to be designed as a loop, wherein this loop can be laid around the median lobe. The above-described denaturation of the tissue and thus the reduction in size of the median lobe is accomplished through the pressure exerted on the tissue of the median lobe by the thread. Since the thread is very flexible, it can adapt to the shape of the median lobe in a very effective manner. The required constriction of the vessels can be accomplished by changing the size of the loop. By enlarging the diameter, the median lobe can be released again and the loop or thread can be removed.

It is furthermore conceivable, according to the invention, that one end of the loop is firmly connected to the implant and the other end is guided loosely to a proximal end of the implant, wherein the size of the diameter of the loop can be varied by pulling on the loose end, and wherein, in particular, the loose end can be fixed releasably to the wire structure during treatment. The fixed connection of one end can be accomplished, for example, by size reduction, adhesive bonding or crimping. The loose end can be moved either manually or by means of an auxiliary instrument. By pulling on the loose end, the loop is tightened, with the result that pressure is exerted on the median lobe. By releasing the loose end, the implant and the thread can be removed from the urinary tract without leaving a residue.

To guide the thread on the wire structure, it is conceivable for at least one or more sleeves, through which the thread, in particular both ends of the loop, can be guided, to be arranged on the wire structure. In this case, the sleeves or a longer hose-like or tube-like sleeve, are passed over a wire of the wire structure. It is conceivable for these sleeves to be fixed at specific positions of the wire, preventing them from sliding along the wire structure. Alternatively, it is also conceivable for such sleeves to be associated with a plurality of wires, thus enabling sleeves of one wire to receive one end of the thread and sleeves of another wire to receive the other end of the thread. By means of these sleeves, the thread can be guided and moved in a very defined manner. Moreover, it is possible to ensure that the thread is guided along the wire structure in a defined manner and does not form a knot with it. Since the wires of the wire structure are pressed against the tissue of the urethra during treatment, it is also possible by means of the sleeves to ensure that the thread can be moved even during treatment in order to change the size of the loop.

A further exemplary embodiment of the invention can provide for a further wire to be arranged on the wire structure, and for this wire to be aligned in such a way that it raises the median lobe during treatment. By means of this raising of the median lobe, the bladder outlet can already be freed for the unobstructed flow of urine. In addition, this raising makes it easier to reach the median lobe with the wire or loop. This wire too can be produced from a shape memory material, in particular Nitinol. The wire unfolds in the urethra in the same way as the wire structure, except that it points in the distal direction and has a rising distal end, with which the median lobe can be pushed up. This further wire can be fastened to the wire structure in the same way as the thread or the wire for constricting or cutting into tissue.

A preferred exemplary embodiment of the present invention is explained in greater detail below with reference to the drawing. In this drawing:

FIG. 1 shows an illustration of a first exemplary embodiment of an implant,

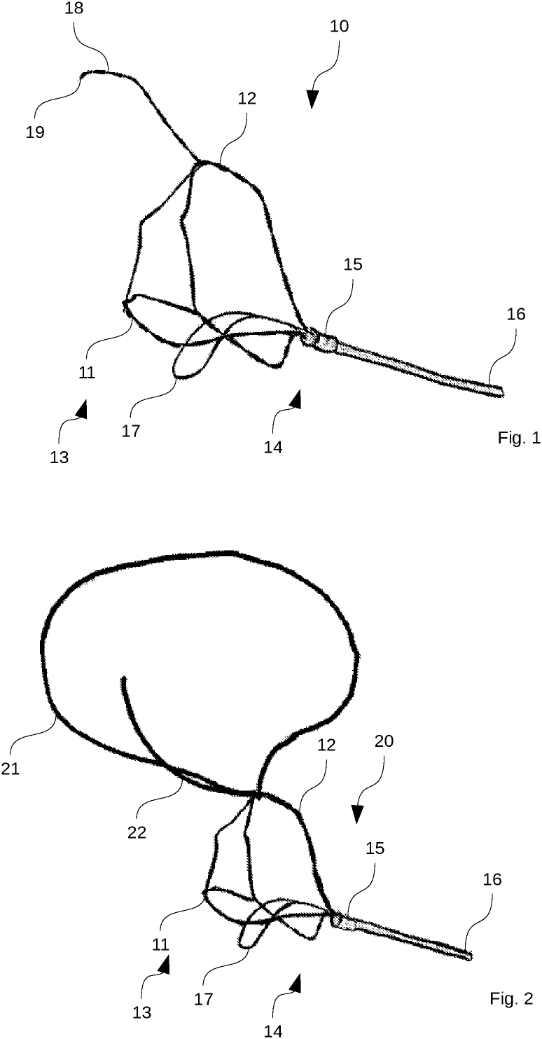

FIG. 2 shows an illustration of a further exemplary embodiment of an implant, and

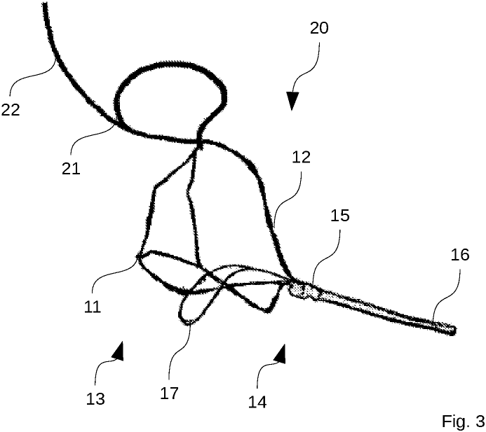

FIG. 3 shows a further illustration of the exemplary embodiment of the implant according to FIG. 2.

Possible exemplary embodiments of the invention are illustrated schematically in the figures. It should be expressly pointed out that there is no intention to restrict the invention to these exemplary embodiments. Rather, it is envisaged that the invention can also be implemented by other embodiments.

FIG. 1 illustrates an implant 10, which consists substantially of a wire structure 11. The wire structure illustrated in FIG. 1 forms a basket, which is formed by three wires 12. In this case, the wires 12 are twisted together in some section or sections. At a distal end 13, the wires 12 are of arcuate design and each run back to a proximal end 14, where they are brought together in a connecting piece 15. This connecting piece 15 positioned at the proximal end 14 is also used to make contact with a handling means 16. This handling means 16 can be designed as a thread or rod or can be part of a catheter.

Furthermore, the implant 10 has an anchoring clamp 17. This anchoring clamp 17 is of arcuate design and serves for anchoring or fixing the implant 10 in the urethra (not illustrated) during treatment. As already stated above, for treatment the implant 10 is introduced into the urethra of the patient through a catheter (not illustrated) in a folded state. At the target location, the implant 10 unfolds into the shape shown in FIG. 1. Here, the wires 12 press on the surrounding tissue of the urethra, resulting in denaturation of the tissue. After treatment, the implant 10 can be pulled back out of the urethra through the catheter, using the handling means 16. During this process, the implant 10 folds back into the catheter. To enable the implant 10 to unfold at the appropriate point in the urethra, it is produced from a shape memory material, namely Nitinol. In addition to this aforementioned material, however, other materials with a shape memory are also conceivable.

The exemplary embodiment illustrated in FIG. 1 now has a further wire 18. This wire 18 is stretched in the distal direction in the unfolded state of the implant 10. In this case, the wire 18 is fastened to one of the wires 12 of the wire structure 11. During placement or in the folded state, this wire 18 is swung inward within the folded wire structure 11. As the implant 10 is unfolded, the wire 18 swings forward in the distal direction and thus forms an angle with the other wires 12. The wire 18 is slightly bent, forming a concave curvature relative to a longitudinal axis of the implant 10. This curvature enables the wire 18 to be placed around the median lobe in a very efficient manner.

The distal end 19 of the wire can be pointed or sharp. During the treatment of the urethra by means of the wire structure 11, the wire 18 presses continuously on the median lobe or tissue (not illustrated). This application of pressure results in denaturation of the tissue. As the implant 10 is pulled out, the distal end 19 of the wire 18 moves along or through the median lobe, and this can result in an incision. Both the denaturation and the incision of the median lobe have the effect that the path is once again clear for the flow of urine.

An alternative exemplary embodiment of the implant 20 illustrated in FIG. 2 envisages that a thread 21 is fastened to at least one of the wires 12. One end of this thread 21 is firmly fastened to the wire structure 11. Another end of the thread 21 is likewise associated with the wire structure 11, but is freely movable. In this case, this free or loose end of the thread 21 is guided along a wire 12 to the proximal end 14 of the implant 20. There, the free end of the thread 21 can be grasped manually or by means of an aid.

According to the invention, provision is made for the loop formed by the thread 21 to be guided around the median lobe and for the tissue of the median lobe to be constricted by pulling on the loose end of the thread 21 (FIG. 3). This constriction can also cause denaturation of the tissue. If necessary, the tensile stress of the thread 21 is regularly increased in order to accelerate the regression of the median lobe. After completion of the treatment, the tensile force on the thread 21 is released. As the implant 20 is pulled out of the patient, the median lobe is also released from the thread 21.

The thread 21 can either be interwoven with the wires 12 of the wire structure 11 or can be guided in sleeves (not illustrated). It is conceivable for a wire 12 to be guided in a sleeve in which the thread 21 can then also be guided. In this case, both the fixed and the loose end of the thread 21 can be guided in this sleeve. Alternatively, it is also conceivable for the fixed end of the thread 21 to be arranged outside the sleeve and for only the loose end of the thread 21 to be guided through the sleeve. Another exemplary embodiment can provide for a plurality of sleeves, which are fixed to the wires 12 and through which the thread 21 is guided with at least one end, to be assigned to a wire 12 of the wire structure 11.

In addition, the exemplary embodiment of the implant 20 according to FIGS. 2 and 3 has a further wire 22. This wire 22 too is likewise fastened to the wire structure 11 and is bent obliquely upward in the distal direction. The wire 22 can preferably consist of a shape memory material and, like the thread 21, is fastened at one end to at least one of the wires 12. In the folded state of the implant 20, this wire 22 too is folded up in the wire structure 11. As the wire structure 11 is unfolded, the wire 22 moves out of the wire structure 11 and serves to press the median lobe upward during treatment, thus ensuring that the latter does not close the bladder opening. Moreover, this pushing of the median lobe upward also serves to place the thread 21 or loop around the median lobe. Owing to the curved shape of the wire 22, the latter has a certain mechanical prestress, by means of which a resilient force can be exerted on the median lobe. After completion of the treatment, the wire 22, together with the implant 20, is pulled out of the urethra.

List of reference signs:

| | |
|---|---|
| 10 | implant |
| 11 | wire structure |
| 12 | wire |
| 13 | distal end |
| 14 | proximal end |
| 15 | connecting piece |
| 16 | handling means |
| 17 | anchoring clamp |
| 18 | wire |
| 19 | distal end |
| 20 | implant |
| 21 | thread |
| 22 | wire |

1. An implant for the treatment of a urinary tract of a person by applying a local ischemic pressure to the tissue of urinary organs by means of a wire structure having at least two wires, wherein the implant can be introduced into the urethra in a folded state with a distal end in the lead and unfolds in the urethra to form the wire structure for the treatment of the tissue, which comprises at least one further means for constricting or cutting into tissue, wherein this means is positioned on the wire structure in such a way that it exerts a local ischemic pressure on a median lobe of the person.

2. The implant as claimed in claim 1, wherein the means for constricting or cutting is a wire made from a shape memory material or is a thread.

3. The implant as claimed in claim 2, wherein the wire can be introduced into the urethra in a folded state relative to the implant and unfolds in the urethra so as to form a predetermined angle with the wire structure.

4. The implant as claimed in claim 1, wherein the means for constricting or cutting is intertwined with a wire of the wire structure.

5. The implant as claimed in claim 2, wherein the wire is bent at least in some section or sections, wherein the wire is bent convexly relative to a longitudinal axis of the implant, thus enabling the bent wire to be laid around the median lobe.

6. The implant as claimed in claim 2, wherein the thread is designed as a loop, wherein this loop can be laid around the median lobe.

7. The implant as claimed in claim 6, wherein a diameter of the loop can be varied.

8. The implant as claimed in claim 6, wherein one end of the loop is firmly connected to the implant and the other end is guided loosely to a proximal end of the implant, wherein the size of the diameter of the loop can be varied by pulling on the loose end of the thread, and wherein, the loose end of the thread can be fixed releasably to the wire structure during treatment.

9. The implant as claimed in claim 6, wherein the thread is guided on the wire structure in at least one sleeve.

10. The implant as claimed in claim 1, wherein a further wire is arranged on the wire structure and this wire is aligned in such a way that it raises the median lobe during treatment.

11. The implant as claimed in claim 1, wherein all the wires and threads are brought together at a proximal end of the implant, and the loose wires and threads can be fixed there or can be led out to the outside through the urethra.
